(19) United States
(12) Patent Application Publication
(10) Pub. No.: US 2018/0245169 A1
(43) Pub. Date: Aug. 30, 2018

(54) NUCLEIC ACID SEQUENCES FOR TYPING DETECTION OF CUTANEOUS HUMAN PAPILLOMAVIRUSES AND USE THEREOF

(71) Applicants: THE FIRST HOSPITAL OF CHINA MEDICIAL UNIVERSITY, Shenyang (CN); Institute of Process Engineering, Chinese Academy of Sciences, Beijing (CN); Zhongke Xinray (Suzhou) biological Science Technologies Co. Ltd, Suzhou (CN)

(72) Inventors: Xinghua Gao, Shenyang (CN); Yuguang Du, Shenyang (CN); Ge Ge, Shenyang (CN); Rui Mao, Shenyang (CN); Ruiqun Qi, Shenyang (CN); Ming Sun, Shenyang (CN); Hong-Duo Chen, Shenyang (CN)

(21) Appl. No.: 15/897,405

(22) Filed: Feb. 15, 2018

(30) Foreign Application Priority Data
Feb. 15, 2017 (CN) 201710081809.5

(51) Int. Cl. C12Q 1/70

(52) U.S. Cl. CPC C12Q 1/708; C12Q 2600/112

(57) ABSTRACT

The present invention belongs to the field of microbial detection, and in particular, relates to nucleic acid sequences for typing detection of cutaneous human papillomaviruses (HPVs) and use thereof. The 30 primer pairs are useful in the preparation of a diagnostic product, for example, a kit, for rapidly detecting the types of cutaneous HPVs. By using the nucleotide sequences, the types of cutaneous HPVs can be rapidly and accurately detected, thereby meeting the requirement for typing detection of cutaneous HPVs in clinic.

### TECHNICAL FIELD

The present invention relates to the field of microbial pathogen detection, and in particular, to nucleic acid sequences for typing detection of cutaneous human papillomaviruses (HPVs) and use thereof.

### BACKGROUND ART

Human papillomaviruses (HPVs) belong to the papillomavirus A genus in the family papovaviridae and are spherical DNA viruses that can cause the proliferation of mucosal squamous cells on human skin, as manifested by common warts and genital warts (condyloma acuminatum), and other symptoms. With the sharp rise in the incidence of condyloma acuminatum among other sexually transmitted diseases and the increased incidence of cervical cancer, anal cancer and so on, HPV infection has received more and more attention.

HPVs are ubiquitous in the natural environment, and are closely related to human health. More and more HPV-related diseases are explored. At present, more than 200 types of HPV have been isolated, and different HPV types can lead to different clinical manifestations. Different types of HPV cause different diseases, the prognoses of these diseases are also different, and even the clinical manifestations of the same disease are also slightly different depending on the type of HPV infected. Detection of the specific type of HPV infected in the patient is of great significance in the screening and diagnosis and in the treatment and prognosis of the disease.

HPVs are associated with a variety of mucocutaneous diseases, including benign and malignant proliferative lesions. HPVs can cause several types of cutaneous diseases such as benign skin warts, solar keratosis (AKs) and non-melanoma skin cancer (NMSCs). The cutaneous HPV types are also frequently detected on healthy human skin. Among the cutaneous HPV infection, the most common disease is skin warts, including common warts, plantar warts and flat warts. Infections with different types of HPV may lead to the occurrence of different skin warts. The common warts are often caused by infection with HPV types 1, 2, and 4. HPV type 2 is the most common type causing common warts on the hands and feet of human. The flat warts are often caused by HPV3, 10, and 28. The skin warts caused by different types of HPV has slightly different clinical manifestations, such as different degrees of keratinization. HPV infection has significant geographical differences. By detecting the type of HPV infection in patients of local region in combination with the analysis of the therapeutic effect, a molecular epidemiological basis is provided for facilitating the implementation of personalized therapeutic regimens for skin warts. However, the vast majority of existing HPV testing methods target mucosal HPVs, and no mature method and nucleic acid sequence for detecting cutaneous HPVs that can be used in clinic are available.

In summary, there is an urgent need for developing a detection method that is practical, accurate, specific and sensitive, so as to meet the requirement for typing detection of cutaneous HPVs in clinic.

### SUMMARY

In view of the above problems, the present invention provides nucleic acid sequences for typing detection of cutaneous human papillomaviruses (HPVs) and use thereof. By using the nucleotide sequences, the types of cutaneous HPVs can be rapidly and accurately detected, thereby meeting the requirement for typing detection of cutaneous HPVs in clinic.

To achieve the above object, the nucleic acid sequences for typing detection of cutaneous HPVs provided in the present invention are specifically nucleic acid sequences of 30 primer pairs that are useful in the typing detection of 30 cutaneous HPVs.

The nucleic acid sequences of the primers are designed and screened as follows. The L1 fragment of each cutaneous HPV is aligned, and the portion with a high difference is selected and used to design a nucleic acid sequence of a primer. 3-10 pairs of candidate primers are designed for each type. Then, a large number of repeated PCR reactions are performed with the multiple pairs of candidate primers using artificially synthesized standard viral nucleic acids (see Table 1 for the Genbank Accession Nos.) and a large number of HPV infected clinical biological samples as templates. The obtained products are sequenced, and the specificity and sensitivity of amplification are validated. Finally, 30 primer pairs with good specificity and sensitivity are obtained, and the nucleotide sequences of which are set forth in SEQ ID Nos: 1 to 60.

The nucleotide sequences of the 30 primer pairs and corresponding HPV types detected are shown in Table 2.

TABLE 1
Genbank Accession Nos. of standard viral nucleic acid sequence

| | Genbank Accession |
|---|---|
| HPV type | No |
| HPV001 | A09292 |
| HPV002 | EF117890 |
| HPV003 | X74462 |
| HPV004 | X70827 |
| HPV005 | M17463 |
| HPV007 | NC_001595 |
| HPV008 | M12737 |
| HPV009 | X74464 |
| HPV010 | NC_001576 |
| HPV012 | X74466 |
| HPV014 | X74467 |
| HPV027 | NC_001584 |
| HPV028 | U31783. |
| HPV029 | U31784 |
| HPV041 | X56147 |
| HPV048 | U31789 |
| HPV049 | NC_001591 |
| HPV050 | U31790 |
| HPV057 | X55965 |
| HPV063 | X70828 |
| HPV065 | X70829 |
| HPV075 | Y15173 |
| HPV076 | Y15174 |
| HPV077 | Y15175 |
| HPV094 | AJ620211 |
| HPV095 | AJ620210 |
| HPV115 | FJ947080 |
| HPV117 | CQ246950 |
| HPV125 | FN547152 |
| HPV160 | AB745694 |

TABLE 2
Nucleotide sequences of 30 primer pairs for detecting the type of HPVs.

| | HPV type | | |
|---|---|---|---|
| SEQ ID No | detected | Primer | Nucleotide sequence |
| SEQ ID No: 1 | HPV1 | Forward primer | GCTGTACTCCTGCTTCAG |
| SEQ ID No: 2 | | Reverse primer | TGCATGTTGCTTGAACAAC |
| SEQ ID No: 3 | HPV2 | Forward primer | CTGCCAGTTTACAGGATACC |
| SEQ ID No: 4 | | Reverse primer | AGACACGGTAGGCATAGC |
| SEQ ID No: 5 | HPV3 | Forward primer | TGGGTTGTACCCCACCTATG |
| SEQ ID No: 6 | | Reverse primer | GCAGGTGGTCTGGCAAAT |
| SEQ ID No: 7 | HPV4 | Forward primer | CCTGCAATAGGTGAACATTG |
| SEQ ID No: 8 | | Reverse primer | GGCCATTTACAAACTGTGG |
| SEQ ID No: 9 | HPV5 | Forward primer | GATCCAAATGTTTATTGTAGGATG |
| SEQ ID No: 10 | | Reverse primer | ATTGACGATGTCTAAACTGAC |
| SEQ ID No: 11 | HPV7 | Forward primer | GAGTGTTTAGAGTACGCTTG |
| SEQ ID No: 12 | | Reverse primer | CAGACGAGTTTTCCACATCT |
| SEQ ID No: 13 | HPV8 | Forward primer | TGTTTTAGCACAAATCAATGC |
| SEQ ID No: 14 | | Reverse primer | CATTCCAGAAGTTAAACTTTGC |
| SEQ ID No: 15 | HPV9 | Forward primer | ACCGTTTGCTAACAGTGG |
| SEQ ID No: 16 | | Reverse primer | GCCTATTTCAATACCTCTACAG |
| SEQ ID No: 17 | HPV10 | Forward primer | CATATTAAAGAGCAACGGTGG |
| SEQ ID No: 18 | | Reverse primer | TCAGAAGGAACACACAAGC |
| SEQ ID No: 19 | HPV12 | Forward primer | CTCAAATAACTATGCCACAGG |
| SEQ ID No: 20 | | Reverse primer | GTCACCATCTTCAATGAAAGTG |
| SEQ ID No: 21 | HPV14 | Forward primer | AGGTATAGAAATAGGCAGAGG |
| SEQ ID No: 22 | | Reverse primer | TTCTACACATGGCAAGGC |
| SEQ ID No: 23 | HPV27 | Forward primer | CCAATAGGTCTGATGTTCCTT |
| SEQ ID No: 24 | | Reverse primer | GGTCCGAGATAGTGGTACT |
| SEQ ID No: 25 | HPV28 | Forward primer | CACAACAGGGAGATTGCC |
| SEQ ID No: 26 | | Reverse primer | AACATGCTGTCGCCATAC |
| SEQ ID No: 27 | HPV29 | Forward primer | ACAGAGTCTCAACCGTTG |
| SEQ ID No: 28 | | Reverse primer | CGTGTCTTCCAAGCTAGTG |
| SEQ ID No: 29 | HPV41 | Forward primer | TACTTTCCTCCATGCTGC |
| SEQ ID No: 30 | | Reverse primer | AACCTCAATCCCACGAAT |
| SEQ ID No: 31 | HPV48 | Forward primer | GAGACTCTGTCTTCTTTTTTGG |
| SEQ ID No: 32 | | Reverse primer | CGTCTAAGCCAATAAGGCC |
| SEQ ID No: 33 | HPV49 | Forward primer | CCTGCAGCAAGTCAACAG |
| SEQ ID No: 34 | | Reverse primer | GCCATCCGTACTTACACTA |
| SEQ ID No: 35 | HPV50 | Forward primer | GGATGCTGATATATTAGCTCATCT |
| SEQ ID No: 36 | | Reverse primer | TTTCTGTAAGGTTGACATTCC |
| SEQ ID No: 37 | HPV57 | Forward primer | CCGGATGAGCTATATGTCAAG |
| SEQ ID No: 38 | | Reverse primer | ACAAAGAGACATTTGTGCTG |
| SEQ ID No: 39 | HPV63 | Forward primer | TTCCTACCCAACCGATCA |
| SEQ ID No: 40 | | Reverse primer | TTATCTCCAAAGGCAAATCG |
| SEQ ID No: 41 | HPV65 | Forward primer | CCATTGGATGTAGTTGCTAC |
| SEQ ID No: 42 | | Reverse primer | ATCCTGACCTTCTTGAGC |
| SEQ ID No: 43 | HPV75 | Forward primer | CCTTAAAATGGCCAATGACA |
| SEQ ID No: 44 | | Reverse primer | CGTGGGAACATAAATAGAGTTG |
| SEQ ID No: 45 | HPV76 | Forward primer | TCCTTACTGTAGGCCACC |
| SEQ ID No: 46 | | Reverse primer | ACCTCTACAGGCCCAAAC |
| SEQ ID No: 47 | HPV77 | Forward primer | TACTACCCCAGGAGACTG |
| SEQ ID No: 48 | | Reverse primer | AAACAGTTGTTCCCGACG |
| SEQ ID No: 49 | HPV94 | Forward primer | GACTTCACTGCATTACAGTT |
| SEQ ID No: 50 | | Reverse primer | CCAACGTTTTGGTCACCA |
| SEQ ID No: 51 | HPV95 | Forward primer | TTCTTCTTTGGCCGAAGG |
| SEQ ID No: 52 | | Reverse primer | CGGTTAAAAAGCTGAGATTCAC |
| SEQ ID No: 53 | HPV115 | Forward primer | ACATACAAAGGACTGACATCT |
| SEQ ID No: 54 | | Reverse primer | GTAGTATCTACCAATGCAAACC |
| SEQ ID No: 55 | HPV117 | Forward primer | CTAGTTCTGTTGGGGACG |
| SEQ ID No: 56 | | Reverse primer | CCACCCAGTCACAAACA |
| SEQ ID No: 57 | HPV125 | Forward primer | CCTGATTATTTGGGCATGG |
| SEQ ID No: 58 | | Reverse primer | GTGTAGGACATATACAGCAC |
| SEQ ID No: 59 | HPV160 | Forward primer | TAGGCCTCAGTGGTCATC |
| SEQ ID No: 60 | | Reverse primer | CAATCACCTGACGTGGAT |

The 30 primer pairs can be used to prepare a diagnostic product for rapid detection of the types of cutaneous HPVs, for example, a kit.

To achieve the above object, the present invention further provides a kit for detecting the types of cutaneous HPVs, which specifically comprises the following components: one of the 30 primer pairs, SYBR Green I, dNTPs, pfu DNA polymerase, and 10× pfu Buffer.

The present invention has the following beneficial effects.

The series of nucleic acid sequences for typing detection of cutaneous human papillomaviruses (HPVs) provided in the present invention have high sensitivity and strong specificity, and can meet the requirement for typing detection of cutaneous HPVs in clinic. Because most of the cutaneous HPVs belong to the Beta genus, and a few belong to the Alpha, Gamma, Mu or Nu genus, the homology between the HPV L1 fragments of different genera is more than 60%, and the homology between some types of HPVs needing typing detection is even as high as 89%, causing a high difficulty in the typing detection of cutaneous HPVs. According to the prior art, it is difficult to screen nucleic acid sequences of primers with suitable specificity and sensitivity for the 30 types of HPVs mentioned in the present invention. However, primers with good specificity and sensitivity are obtained via screening in the present invention after a large number of experimental tests, thereby achieving the typing detection of cutaneous HPV in clinic. In order to obtain primers with suitable nucleotide sequences, more than two years of research efforts and nearly one million research funds are spent in designing, screening and sequencing nucleic acid sequences of primers, and validating the specificity and sensitivity of amplification. The results show that by using the series of nucleic acid sequences of the primers, test results with high accuracy and sensitivity are obtained.

In the present invention, 30 pairs of nucleic acid sequences for typing detection of cutaneous HPVs are constructed, and the types detected cover all types that are needed in the clinic, thereby filling a gap in the method for typing detection of cutaneous HPV in clinical application.

### BRIEF DESCRIPTION OF THE DRAWINGS

FIG. 1 shows primer screening curves.

FIG. 2 shows reaction curves for the sensitivity and specificity detection of primers.

FIG. 3 shows electrophoresis of PCR products, in which Lane 1: Sample 1; Lane 2: Sample 2; Lane 3: Sample 3; Lane 4: Sample 4; Lane 5: Sample 5; Lane 6: Sample 6; Lane 7: Sample 7; Lane 8: Sample 8; and Lane 9: Sample 9.

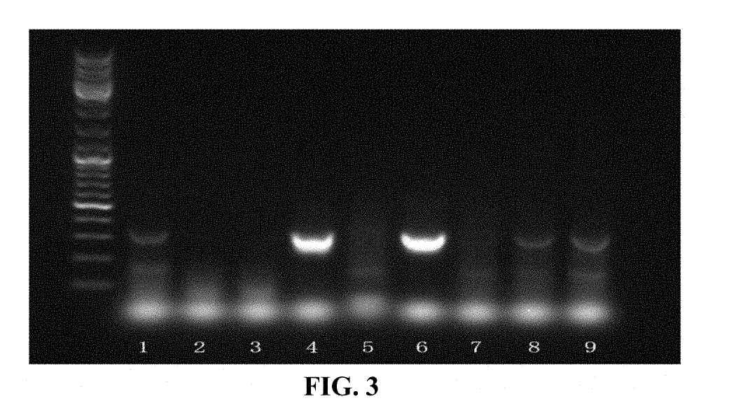

### DETAILED DESCRIPTION

The present invention will be further described with reference to the following specific examples, which are intended to facilitate the understanding of the present invention. However, these examples are merely provided for the purpose of illustrating the present invention, and the present invention is not limited thereto. The operations and methods that are not particularly described in the examples are all conventional operations and methods in the art.

### EXAMPLE 1

Design and screening of nucleic acid sequences of the primers: The L1 fragment of each cutaneous HPV was aligned, and the portion with a high difference was selected and used to design a nucleic acid sequence of a primer. Multiple pairs of candidate primers are designed for each type. Then, a large number of repeated PCR reactions were performed with the multiple pairs of candidate primers using artificially synthesized standard viral nucleic acids and a large number of HPV infected clinical biological samples as templates. The obtained products were sequenced, and the specificity and sensitivity of amplification were validated. Finally, 30 primer pairs with good specificity and sensitivity were obtained, and the nucleotide sequences of which were set forth in SEQ ID Nos: 1 to 60.

Specifically, the screening method comprised the following steps.

(1) One candidate primer pair was randomly selected and subjected to an amplification reaction in a reaction system of 25 μl in a centrifuge tube. The reaction system comprised specifically each 0.2 μM of a forward primer and a reverse primer, SYBR Green I, 0.8 μM of dNTP, 2.5 U of pfu DNA polymerase, 5 μl of 10× pfu Buffer, and 1 ng/μl of plasmid DNA. Meanwhile, a negative control group was set, in which the components were the same as those in the above reaction system, except that the plasmid DNA was replaced by ddH₂O. The centrifuge tube was placed in an ABI Step One real time PCR Instrument, pre-denatured for 5 min at a temperature set to 94° C., and then subjected to 40 cycles of 50 s at 94° C., 50 s at 49° C., and 1 min at 72° C., followed by 5 min at 72° C. The resulting amplification curve or agarose gel electrophoretogram was observed to examine whether a desirable product exists. The HPV type in the sample was known, and thus primers having a nucleotide sequence set forth in SEQ ID No: 1 to 60 were preliminarily screened out by using the method.

(2) Detection of sensitivity and specificity of the screened primer pairs for 30 HPV types: The nucleic acid sequences of a primer pair for one HPV type were respectively reacted with various concentrations of plasmid DNA of this type and the plasmid DNAs of other HPV types. The reaction rate and the presence of cross-reactions were observed. The primer pair for each HPV type were examined with 8 groups of reaction system that was the same as the reaction system in the step 1, where 1 μL of various concentrations of plasmid DNA (1 ng/μl, 10-fold dilution of the plasmid DNA, and 2²⁰-fold dilution of the plasmid DNA) of this type was respectively added to 3 groups of reaction system, 1 ng/μl plasmid DNA of other HPV types was added respectively to 4 groups of reaction system, and ddH₂O was added in place of the plasmid DNA to 1 group of reaction system as a negative control group. The reaction was carried out in a fluorescence PCR instrument, amplification was observed, and the best primer pair with which the plasmid DNA at various concentrations of this type could be amplified without any cross-reaction, that is, the primer pair with high specificity and sensitivity, was screened out. Therefore, the nucleic acid sequences of the primers for 30 HPV types were ensured to have a high amplification efficiency while no cross-reactions exist.

### EXAMPLE 2

Detection method with nucleic acid sequences of candidate primer pair for detecting HPV type 1

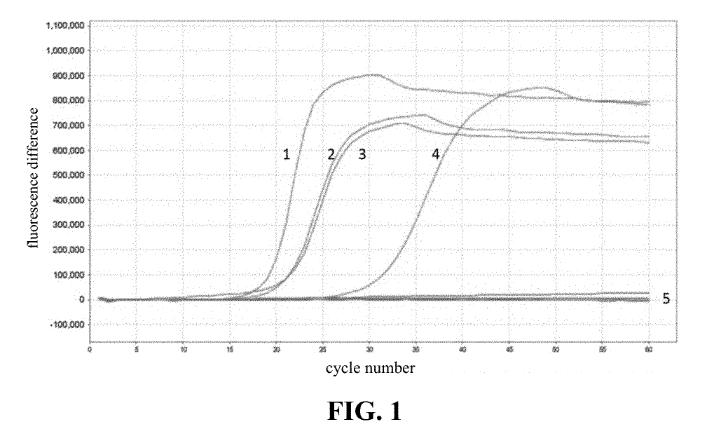

(1) One candidate primer pair for HPV type 1 was randomly selected, and subjected to an amplification reaction in a reaction system of 25 μl in a centrifuge tube. The reaction system comprised specifically each 0.2 μM of a forward primer and a reverse primer, SYBR Green I, 0.8 μM of dNTP, 2.5 U of pfu DNA polymerase, 5 μl of 10× pfu Buffer, and 1 ng/μl of plasmid DNA. Meanwhile, a negative control group was set, in which the components were the same as those in the above reaction system, except that the plasmid DNA was replaced by ddH₂O. The centrifuge tube was placed in a fluorescence PCR instrument for reacting, pre-denatured for 5 min at a temperature set to 94° C., and then subjected to 40 cycles of 50 s at 94° C., 50 s at 49° C., and 1 min at 72° C., followed by 5 min at 72° C. Then, 3 candidate primer pairs were additionally selected, with which the amplification reaction was repeated. The resulting amplification and primer screening curve are shown in FIG. 1, in which curve 1: amplification curve with a first primer pair for HPV type 1; curve 2: amplification curve with a second primer pair for HPV type 1; curve 3: amplification curve with a third primer pair for HPV type 1; curve 4: amplification curve with a fourth primer pair for HPV type 1; and curve 5: negative control group without plasmid DNA. The type of HPV1 in the sample was known, and thus primers having a nucleotide sequence set forth in SEQ ID No: 1 and SEQ ID No: 2 were screened out.

(2) Detection of sensitivity and specificity of the screened primer pairs for HPV type 1: The nucleic acid sequences of a primer pair for one HPV type were respectively reacted with various concentrations of plasmid DNA of this type and the plasmid DNAs of other HPV types. The reaction rate and the presence of cross-reactions were observed. The primer pair for each HPV type were examined with 8 groups of reaction system that was the same as the reaction system in the step 1, where various concentrations of plasmid DNA (1 ng/μl, 10-fold dilution of the plasmid DNA, and 2²⁰-fold dilution of the plasmid DNA) of this type was respectively added to 3 groups of reaction system, 1 ng/μl of plasmid DNA of other HPV types was added respectively to 4 groups of reaction system, and ddH₂O was added in place of the plasmid DNA to 1 group of reaction system as a negative control group. The reaction was carried out in a fluorescence PCR instrument, the amplification was observed, and the best primer pair with which the plasmid DNA at various concentrations of this type could be amplified without any cross-reaction, that is, the primer pair with high specificity and sensitivity, was screened out. Therefore, the nucleic acid sequences of the primers for 30 HPV types were ensured to have a high amplification efficiency while no cross-reactions exist.

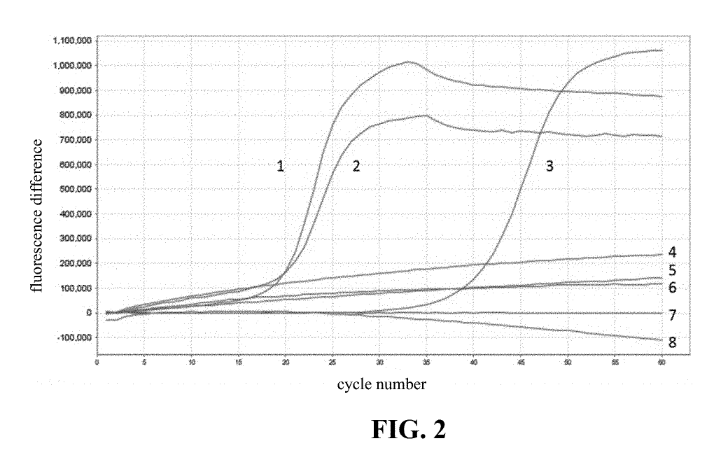

The reaction curves for detecting the sensitivity and specificity of the primer are shown in FIG. 2, in which curve 1: amplification curve with the primer sequence for HPV1 when the concentration of the HPV1 plasmid was 1 ng/μl ; curve 2: amplification curve with the primer sequence for HPV1 when the concentration of the HPV1 plasmid DNA was 2-fold diluted; curve 3: amplification curve with the primer sequence for HPV1 when the concentration of the HPV1 plasmid was 2²⁰-fold diluted; curve 4: amplification curve with the primer sequence for HPV1 when HPV2, HPV3, HPV4, HPV5, HPV7, HPV8, and HPV9 plasmid DNAs were added; curve 5: amplification curve with the primer sequence for HPV1 when HPV10, HPV12, HPV14, HPV27, HPV28, HPV29, and HPV41 plasmid DNAs were added; curve 6: amplification curve with the primer sequence for HPV1 when HPV48, HPV49, HPV50, HPV57, HPV63, HPV65, and HPV75 plasmid DNAs were added; curve 7: amplification curve with the primer sequence for HPV1 when HPV76, HPV77, HPV94, HPV95, HPV115, HPV117, HPV125, and HPV160 plasmid DNAs were added; and curve 8: negative control group without plasmid DNAs.

I. Detection of the HPV Type in Clinical Sample

Use of the nucleic acid sequences of the screened primer pair in the detection of the HPV type in clinical samples of skin warts

The samples were derived from dermatological outpatients with skin warts from the First Affiliated Hospital of China Medical University, and 30 clinical samples of skin warts were collected. DNA was extracted from the sample using a plasmid DNA Mini Extraction Kit, and about 1 ng of clinical sample DNA was added to a reaction system that was the same as the reaction system in the step 1 of Example 1. The sample was sequenced by using nucleic acid sequences of a universal primer pair for cutaneous HPVs, as shown in 3, in which Lane 1: electrophoretic band of the amplification product of Sample 1; Lane 2: electrophoretic band of the amplification product of Sample 2; Lane 3: electrophoretic band of the amplification product of Sample 3; Lane 4: electrophoretic band of the amplification product of Sample 4; Lane 5: electrophoretic band of the amplification product of Sample 5; Lane 6: electrophoretic band of the amplification product of Sample 6; Lane 7: electrophoretic band of the amplification product of Sample 7; Lane 8: electrophoretic band of the amplification product of Sample 8; and Lane 9: electrophoretic band of the amplification product of Sample 9. As can be seen from the comparison result of the HPV types detected in the samples by the two detection methods, the detection accuracy with the nucleic acid sequence of the present invention was 100%, and the sensitivity was higher. The detection results are shown in Table 3.

TABLE 3
Comparison of the detection results of the two methods

| | | Detection |
|---|---|---|
| | Detection with | with a |
| Clinical | the present | universal |
| sample | nucleic acid | primer |
| No. | sequence | pair |
| 1 | HPV27 | HPV27 |
| 2 | HPV27 | Not |
| | | detected |
| 3 | HPV27 | Not |
| | | detected |
| 4 | HPV1 | HPV1 |
| 5 | HPV27 | Not |
| | | detected |
| 6 | HPV27 | HPV27 |
| 7 | HPV1 | Not |
| | | detected |
| 8 | HPV27 | HPV27 |
| 9 | HPV57 | HPV57 |
| 10 | HPV57 | HPV57 |
| 11 | HPV1 | HPV1 |
| 12 | HPV2, HPV27 | HPV27 |
| 13 | HPV27 | HPV27 |
| 14 | HPV57 | HPV57 |
| 15 | HPV27 | Not |
| | | detected |
| 16 | HPV2 | HPV2 |
| 17 | HPV57 | HPV57 |
| 18 | HPV1 | Not |
| | | detected |
| 19 | HPV27 | Not |
| | | detected |
| 20 | HPV27 | HPV27 |
| 21 | HPV57 | HPV57 |
| 22 | HPV1 | HPV1 |
| 23 | HPV1 | HPV1 |
| 24 | HPV4 | Not |
| | | detected |
| 25 | HPV27 | Not |
| | | detected |
| 26 | HPV27 | HPV27 |
| 27 | HPV57 | HPV57 |
| 28 | HPV2 | Not |
| | | detected |
| 29 | HPV2 | Not |
| | | detected |
| 30 | HPV27 | HPV27 |

The detection results show that the detection results with the nucleic acid sequences of the present invention are more accurate than the detection results obtained with a universal primer pair.

1. Nucleic acid sequences for typing detection of cutaneous human papillomaviruses (HPVs), wherein the nucleic acid sequences are specifically nucleic acid sequences of 30 primer pairs that are useful in the typing detection of cutaneous HPVs, and the nucleic acid sequences of the primer pairs are set forth in SEQ ID NOs: 1 to 60.

2. The nucleic acid sequences of 30 primer pairs according to claim 1, which are useful in the preparation of a diagnostic product for rapidly detecting the types of cutaneous HPVs.

3. The diagnostic product for rapidly detecting the types of cutaneous HPVs according to claim 2, wherein the diagnostic product is a kit.

4. The nucleic acid sequences of 30 primer pairs according to claim 2, wherein the kit specifically comprises the following components: one of the 30 primer pairs, SYBR Green I, dNTPs, pfu DNA polymerase, and 10× pfu Buffer.

5. The nucleic acid sequences of 30 primer pairs according to claim 4, wherein the components in the kit are specifically 0.2 μm of each primer in the primer pairs, 120 μm of SYBR, 0.8 μm of dNTP, 2.5 U of pfu DNA polymerase, and 5 μl of 10× pfu Buffer.


SEQUENCE LISTING (60 sequences)

SEQ ID NO: 1 - DNA, 18 bp, Artificial sequence
  HPV1 forward primer
gctgtactcctgcttcag

SEQ ID NO: 2 - DNA, 19 bp, Artificial sequence
  HPV1 reverse primer
tgcatgttgcttgaacaac

SEQ ID NO: 3 - DNA, 20 bp, Artificial sequence
  HPV2 forward primer
ctgccagtttacaggatacc

SEQ ID NO: 4 - DNA, 18 bp, Artificial sequence
  HPV2 reverse primer
agacacggtaggcatagc

SEQ ID NO: 5 - DNA, 20 bp, Artificial sequence
  HPV3 forward primer
tgggttgtaccccacctatg

SEQ ID NO: 6 - DNA, 18 bp, Artificial sequence
  HPV3 reverse primer
gcaggtggtctggcaaat

SEQ ID NO: 7 - DNA, 20 bp, Artificial sequence
  HPV4 forward primer
cctgcaataggtgaacattg

SEQ ID NO: 8 - DNA, 19 bp, Artificial sequence
  HPV4 reverse primer
ggccatttacaaactgtgg

SEQ ID NO: 9 - DNA, 24 bp, Artificial sequence
  HPV5 forward primer
gatccaaatgtttattgtaggatg

SEQ ID NO: 10 - DNA, 21 bp, Artificial sequence
  HPV5 reverse primer
attgacgatgtctaaactgac

SEQ ID NO: 11 - DNA, 20 bp, Artificial sequence
  HPV7 forward primer
gagtgtttagagtacgcttg

SEQ ID NO: 12 - DNA, 20 bp, Artificial sequence
  HPV7 reverse primer
cagacgagttttccacatct

SEQ ID NO: 13 - DNA, 21 bp, Artificial sequence
  HPV8 forward primer
tgttttagcacaaatcaatgc

SEQ ID NO: 14 - DNA, 22 bp, Artificial sequence
  HPV8 reverse primer
cattccagaagttaaactttgc

SEQ ID NO: 15 - DNA, 18 bp, Artificial sequence
  HPV9 forward primer
accgtttgctaacagtgg

SEQ ID NO: 16 - DNA, 22 bp, Artificial sequence
  HPV9 reverse primer
gcctatttcaatacctctacag

SEQ ID NO: 17 - DNA, 21 bp, Artificial sequence
  HPV10 forward primer
catattaaagagcaacggtgg

SEQ ID NO: 18 - DNA, 19 bp, Artificial sequence
  HPV10 reverse primer
tcagaaggaacacacaagc

SEQ ID NO: 19 - DNA, 21 bp, Artificial sequence
  HPV12 forward primer
ctcaaataactatgccacagg

SEQ ID NO: 20 - DNA, 22 bp, Artificial sequence
  HPV12 reverse primer
gtcaccatcttcaatgaaagtg

SEQ ID NO: 21 - DNA, 21 bp, Artificial sequence
  HPV14 forward primer
aggtatagaaataggcagagg

SEQ ID NO: 22 - DNA, 18 bp, Artificial sequence
  HPV14 reverse primer
ttctacacatggcaaggc

SEQ ID NO: 23 - DNA, 21 bp, Artificial sequence
  HPV27 forward primer
ccaataggtctgatgttcctt

SEQ ID NO: 24 - DNA, 19 bp, Artificial sequence
  HPV27 reverse primer
ggtccgagatagtggtact

SEQ ID NO: 25 - DNA, 18 bp, Artificial sequence
  HPV28 forward primer
cacaacagggagattgcc

SEQ ID NO: 26 - DNA, 18 bp, Artificial sequence
  HPV28 reverse primer
aacatgctgtcgccatac

SEQ ID NO: 27 - DNA, 18 bp, Artificial sequence
  HPV29 forward primer
acagagtctcaaccgttg

SEQ ID NO: 28 - DNA, 19 bp, Artificial sequence
  HPV29 reverse primer
cgtgtcttccaagctagtg

SEQ ID NO: 29 - DNA, 18 bp, Artificial sequence
  HPV41 forward primer
tactttcctccatgctgc

SEQ ID NO: 30 - DNA, 18 bp, Artificial sequence
  HPV41 reverse primer
aacctcaatcccacgaat

SEQ ID NO: 31 - DNA, 22 bp, Artificial sequence
  HPV48 forward primer
gagactctgtcttcttttttgg

SEQ ID NO: 32 - DNA, 19 bp, Artificial sequence
  HPV48 reverse primer
cgtctaagccaataaggcc

SEQ ID NO: 33 - DNA, 18 bp, Artificial sequence
  HPV49 forward primer
cctgcagcaagtcaacag

SEQ ID NO: 34 - DNA, 19 bp, Artificial sequence
  HPV49 reverse primer
gccatccgtacttacacta

SEQ ID NO: 35 - DNA, 24 bp, Artificial sequence
  HPV50 forward primer
ggatgctgatatattagctcatct

SEQ ID NO: 36 - DNA, 21 bp, Artificial sequence
  HPV50 reverse primer
tttctgtaaggttgacattcc

SEQ ID NO: 37 - DNA, 21 bp, Artificial sequence
  HPV57 forward primer
ccggatgagctatatgtcaag

SEQ ID NO: 38 - DNA, 20 bp, Artificial sequence
  HPV57 reverse primer
acaaagagacatttgtgctg

SEQ ID NO: 39 - DNA, 18 bp, Artificial sequence
  HPV63 forward primer
ttcctacccaaccgatca

SEQ ID NO: 40 - DNA, 20 bp, Artificial sequence
  HPV63 reverse primer
ttatctccaaaggcaaatcg

SEQ ID NO: 41 - DNA, 20 bp, Artificial sequence
  HPV65 forward primer
ccattggatgtagttgctac

SEQ ID NO: 42 - DNA, 18 bp, Artificial sequence
  HPV65 reverse primer
atcctgaccttcttgagc

SEQ ID NO: 43 - DNA, 20 bp, Artificial sequence
  HPV75 forward primer
ccttaaaatggccaatgaca

SEQ ID NO: 44 - DNA, 22 bp, Artificial sequence
  HPV75 reverse primer
cgtgggaacataaatagagttg

SEQ ID NO: 45 - DNA, 18 bp, Artificial sequence
  HPV76 forward primer
tccttactgtaggccacc

SEQ ID NO: 46 - DNA, 18 bp, Artificial sequence
  HPV76 reverse primer
acctctacaggcccaaac

SEQ ID NO: 47 - DNA, 18 bp, Artificial sequence
  HPV77 forward primer
tactaccccaggagactg

SEQ ID NO: 48 - DNA, 18 bp, Artificial sequence
  HPV77 reverse primer
aaacagttgttcccgacg

SEQ ID NO: 49 - DNA, 20 bp, Artificial sequence
  HPV94 forward primer
gacttcactgcattacagtt

SEQ ID NO: 50 - DNA, 18 bp, Artificial sequence
  HPV94 reverse primer
ccaacgttttggtcacca

SEQ ID NO: 51 - DNA, 18 bp, Artificial sequence
  HPV95 forward primer
ttcttctttggccgaagg

SEQ ID NO: 52 - DNA, 22 bp, Artificial sequence
  HPV95 reverse primer
cggttaaaaagctgagattcac

SEQ ID NO: 53 - DNA, 21 bp, Artificial sequence
  HPV115 forward primer
acatacaaaggactgacatct

SEQ ID NO: 54 - DNA, 22 bp, Artificial sequence
  HPV115 reverse primer
gtagtatctaccaatgcaaacc

SEQ ID NO: 55 - DNA, 18 bp, Artificial sequence
  HPV117 forward primer
ctagttctgttggggacg

SEQ ID NO: 56 - DNA, 18 bp, Artificial sequence
  HPV117 reverse primer
ccaccacagtcacaaaca

SEQ ID NO: 57 - DNA, 19 bp, Artificial sequence
  HPV125 forward primer
cctgattatttgggcatgg

SEQ ID NO: 58 - DNA, 21 bp, Artificial sequence
  HPV125 reverse primer
gtgtaggacaatatacagcac

SEQ ID NO: 59 - DNA, 18 bp, Artificial sequence
  HPV160 forward primer
taggcctcagtggtcatc

SEQ ID NO: 60 - DNA, 18 bp, Artificial sequence
  HPV160 reverse primer
caatcacctgacgtggat